(19) United States
(12) Patent Application Publication
(10) Pub. No.: US 2020/0085996 A1
(43) Pub. Date: Mar. 19, 2020

(54) RESORBABLE MATERIAL DISPENSER AND METHOD OF USE

(71) Applicant: David B. Powers, Durham, NC (US)

(72) Inventor: David B. Powers, Durham, NC (US)

(21) Appl. No.: 16/616,786

(22) PCT Filed: May 23, 2018
(86) PCT No.: PCT/US18/34202

§ 371 Date: Nov. 25, 2019

Related U.S. Application Data
(60) Provisional application No. 62/510,354, filed on May 24, 2017.

(51) Int. Cl. A61L 24/00; A61B 17/00; A61L 24/04; A61L 27/18; A61L 27/58; A61B 17/88

(52) U.S. Cl. CPC A61L 24/0042; A61B 17/00491; A61L 24/046; A61B 17/8805; A61L 27/58; A61B 17/8836; A61L 27/18

(57) ABSTRACT

A method of stabilizing a craniomaxillofacial fracture includes heating a resorbable material to make the resorbable material flowable; applying the flowable resorbable material to a portion of a first bone fragment defining a fracture; and bonding the portion of the first bone fragment to an adjacent second bone fragment to stabilize the first bone fragment relative to the second bone fragment; wherein the first bone fragment is stabilized relative to the second bone fragment without an external force being applied directly to either the first bone fragment or the second bone fragment.

### CROSS-REFERENCE TO RELATED APPLICATIONS

This application claims the benefit of U.S. Provisional Patent Application No. 62/510,354, filed on May 24, 2017, the contents of which are incorporated herein by reference in their entirety.

### BACKGROUND

Current treatments for craniomaxillofacial fractures involve fixing adjacent bone fragments defining a facture site using metal or biodegradable plates and screws. During such treatment procedures, the plates must be contoured and shaped to the unique geometries of the individual bone fragments. Although such treatments have generally been successful, such treatment of bone fractures via plates is time consuming and requires great care and skill, requiring the practitioner to carefully select and delicately attach bone plates and screws to bone fragments without causing any further damage or dislocation to the fracture site. Moreover, the bones of some patients, e.g. children, the elderly, etc., may often be too weak and fragile for the attachment of bone plates and screws to individual, discrete bone fragments defining a fractured bone site. Accordingly, there is a need for a device and method for repairing craniomaxillofacial fractures that is easy and quick to use, which does not require applying any external force or pressure to the bone fragments of the fracture site (e.g. which does not involve fixation of any attachment elements directly onto and/or through the bone fragment), which can provide a stable and structurally sound interconnected bone structure to which bone plates and screws may subsequently easily and ready be applied, and which does not face any other obstacles plaguing conventional bone plate and screw methods of treating bone fractures.

### SUMMARY

In one aspect, a method of stabilizing a craniomaxillofacial fracture is provided. The method includes heating a resorbable material to make the resorbable material flowable. The flowable resorbable material is applied to a portion of a first bone fragment defining a fracture. The portion of the first bone fragment is bonded to an adjacent second bone fragment to stabilize the first bone fragment relative to the second bone fragment. The first bone fragment is stabilized relative to the second bone fragment without the application of an external force applied directly to either the first bone fragment or the second bone fragment.

in some embodiments, the first bone fragment is bonded and stabilized relative to the second bone fragment without the use of any fasteners or additional attachment elements. In some embodiments, a bone plate is attached to the first bone fragment and the second bone fragment following the bonding of the first bone fragment to the second fragment with the flowable resorbable material. In some embodiments, the bone plate is attached to the first bone fragment by a first screw. In some embodiments, the bone plate is attached to the second bone fragment by a second screw.

In some embodiments, the resorbable material is applied via a resorbable material dispensing applicator to an edge of the first bone fragment. In some embodiments, the dispensing applicator includes a feeder element configured to move a solid piece of a resorbable material past a heating element and into a nozzle. In some embodiments, the feeder element is configured to move the resorbable material in response to the activation of the dispensing applicator via a trigger. In some embodiments, the nozzle includes a valve assembly configured to prevent flow of melted resorbable material when the trigger is released.

In another aspect, a method applying a resorbable material to a craniomaxillofacial fracture is provided. A resorbable material dispensing applicator is provided. The dispensing applicator includes a housing body within which is located a motor, a drive element configured to be activated by the motor, a feeder element configured to move in response to activation of the drive element, and a heating element. A solid piece of resorbable material is loaded into the dispensing applicator. The feeder element is activated to translate the solid piece of resorbable material relative to the heating element to melt the solid piece of resorbable material. The melted resorbable material is dispensed through a nozzle of the dispensing applicator to a first bone fragment.

In some embodiments, the applied melted resorbable material adheres the first bone fragment to an adjacent bone fragment. In some embodiments, the melted resorbable material is applied to an edge of the first bone fragment. In some embodiments, the applicator is reusable.

In some embodiments, the feeder element is configured to rotate in response to activation of the drive element. The rotation of the feeder element is configured to urge the solid piece of resorbable material towards the heating element and nozzle. In some embodiments, a pinch roller is configured to urge the solid piece of resorbable material into contact with the feeder element. In some embodiments, the resorbable material is one of poly-D and L-lactic acid, L-lactide-co-glycolide, an admixture of polylactic acid and an admixture of polyglycolide acid.

In a further aspect, a resorbable biomaterial dispensing applicator configured to dispense melted resorbable biomaterial to a craniomaxillofacial fracture is provided. The resorbable biomaterial dispensing applicator includes a housing body defining a motor, a drive element, a heating element, a feeder element and a nozzle. The drive element is configured to be activated by the motor. The heating element defines a passageway therethrough. The heating element is configured to melt a resorbable material stick. A feeder element is configured to move in response to activation of the drive element. The movement of the feeder element configured to translate the resorbable material stick into the passageway of the heating element. A nozzle defines a passageway extending between an outlet of the heating element passageway and an outlet opening. The nozzle is defined by a cross-sectional area of between approximately 0.25 mm and approximately 5 mm and a length of between approximately 10 mm and approximately 300 mm. The nozzle is configured to allow the heated and melted resorbable material to be applied to an edge of a bone fragment forming a part of the craniomaxillofacial fracture.

In some embodiments, a resorbable material stick is loaded within the housing body. In some embodiments, a temperature sensor is configured to prevent movement of the feeder element in response to a sensed temperature, the melted bioresorbable material within the housing body being greater than a predetermined threshold temperature. In some embodiments, the resorbable material stick is entirely enclosed by the housing body when the resorbable material stick is loaded within the housing body.

### BRIEF DESCRIPTION OF THE DRAWINGS

FIG. 1 is a cross-sectional view of a resorbable material applicator, according to one embodiment.

FIG. 2 is a side view of the resorbable material applicator of FIG. 1 shown in an open configuration, according to one embodiment.

FIG. 3A is a cross-sectional view of a resorbable material applicator, according to one embodiment.

FIG. 3B is a side view of the resorbable material applicator of FIG. 3A shown in an open configuration, according to one embodiment.

FIG. 4A is a partial cross-sectional view of the nozzle in a closed configuration, according to one embodiment.

FIG. 4B is a partial cross-section view of the nozzle of FIG. 4A in an open configuration, according to one embodiment.

FIGS. 5A, 5B, 5C, and 5D illustrate nozzle attachments according to various embodiments.

FIG. 6 illustrates a craniomaxillofacial fracture pattern according to one embodiment.

FIG. 7 illustrates a resorbable material applicator being used to treat a craniomaxillofacial fracture, according to one embodiment.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” or “approximately” will be understood by persons of ordinary skill in the art and will vary to some extent depending upon the context in which it is used. If there are uses of the term which are not clear to persons of ordinary skill in the art, given the context in which it is used, “about” or “approximately” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

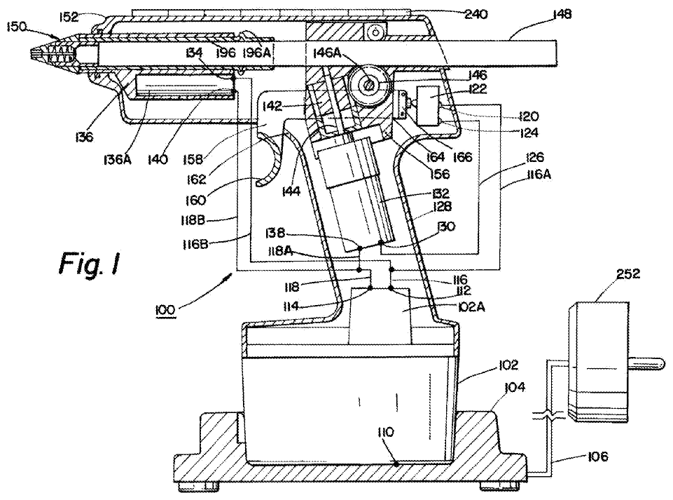

Referring to FIG. 1, a resorbable material applicator 100 according to one embodiment is shown. In general, the resorbable material applicator 100 includes a housing 128, within which a motor 132 and a heating element 136 are housed. The housing 128 is configured to receive a resorbable material stick 148 that is configured to be melted by the heating element 136, with the melted resorbable material being configured to be dispensed via a nozzle 150 of the housing 128. The resorbable material applicator 100 may be corded or cordless (e.g. powered via a rechargeable battery). In some embodiments, some or all of the components of the resorbable material applicator 100 may be configured for single-use, with any non-disposable components of the resorbable material applicator 100 being configured to be capable of being sterilized prior to a subsequent use of the resorbable material applicator 100.

The resorbable material used with and delivered by the resorbable material applicator 100 may include any number of know resorbable biomaterials. Non-limiting examples of resorbable biomaterials that may be used include, e.g. Poly-D and L-Lactic acid (PDDLA)—sold as SonicWeld by KLS Martin (Jacksonville, Fla.); L-lactide-co-glycolide—sold as RAPIDSORB by DePuy Synthes (West Chester, Pa.); as well as other admixtures of Polylactic Acid (PLA) and Polyglycolic Acid (PGA).

As shown in FIG. 1, movement of the resorbable material stick 148 though the heating element 136 may be accomplished by a feed roller 146 that urges the resorbable material stick 148 towards the heating element and into the nozzle 150 in response to the feed roller 146 being rotated by a gear operably attached to a drive shaft 144 of the motor 132. An optionally provided pinch roller assembly 154 may be configured to press the resorbable material stick 148 against the feed roller 146 to assist in urging the resorbable material stick 148 toward the nozzle assembly 150 upon the activation of the feed roller 146. As will be understood, according to other embodiments, the resorbable material stick 148 may be moved through the heating element 136 and into the nozzle 150 according to any number of different arrangements, such as, e.g. a plunger arrangement (not shown).

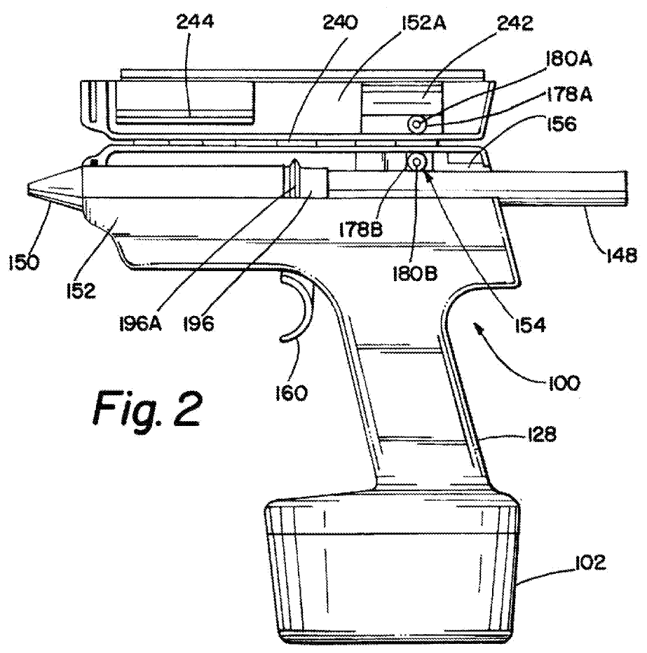

In some embodiments, the resorbable material stick 148 may be loaded directly into a passageway 137 extending through heating element 136. Alternatively, in other embodiments, the loading of the resorbable material stick 148 into the passageway 137 and/or the sterile reuse of the resorbable material applicator 100 may be facilitated by providing the resorbable material stick 148 within a removable holder 196 that is turn in turn loaded into passageway 137, such as, e.g. illustrated by the embodiment of FIG. 1. When loaded within the housing 128, a front end of the removable holder 196 is adapted to engage a rear opening of the nozzle 156, and a peripheral stop ring 197 formed about a rear end of the removable holder 196 is configured to abut a rear opening of the passageway 137 of the heating element 136, such that upon activation of the feed roller 146, the resorbable material stick which is loaded into the removable holder 196 is urged relative to the removable holder 196 towards the nozzle 150 while the removable holder 196 remains stationary. In some embodiments, the housing 128 may be configured to be reusable, with the housing 128 including a hinged connection, such as, e.g. illustrated by the embodiment of FIG. 2, via which a new removable holder 196 and resorbable material stick 148 may be loaded as needed.

As shown in FIGS. 1 and 2, according to some embodiments, a rear portion of the housing 128 may be formed with an opening 129 through which a rear end of the resorbable material stick 148 extends upon initial loading of the resorbable material stick 148 into the housing 128. By providing an opening 129 through the housing 128, a long resorbable material stick 148 may be loaded into the housing 128, allowing for a larger volume of resorbable material to be dispensed upon a single loading of a single resorbable material stick 148 and optional removable holder 196 into the housing 128. Additionally, according to some embodiments, if additional resorbable material is needed during use of the resorbable material applicator 100, additional resorbable material sticks 148 may be fed directly through the opening 129 in the housing 128, allowing for continuous use of the resorbable material applicator 100 as the source of resorbable material is replenished.

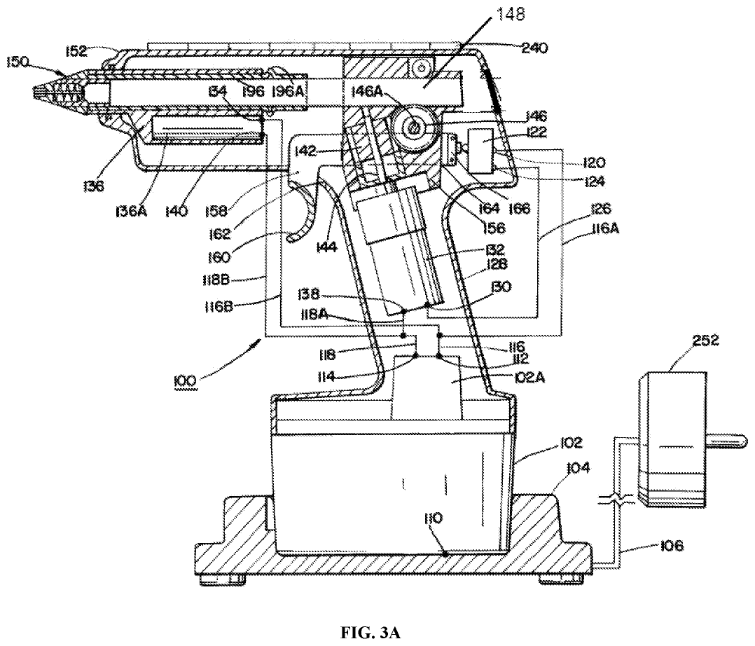

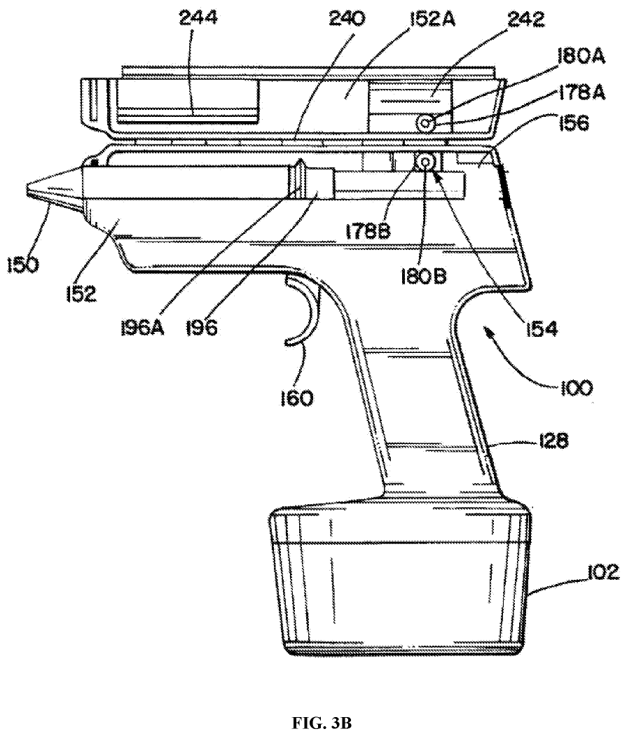

Alternatively, it may be desirable to maintain the resorbable material as sterile as possible prior to, and during, use of the resorbable material applicator 100. As such, in some embodiments, it may be desirable to minimize the exposure of the resorbable material stick 148 to the ambient atmosphere by providing an enclosed housing body 128. According to some such embodiments, the housing 128 may be substantially similar to that of the housing of the resorbable material applicator 100 illustrated in FIGS. 1 and 2, with the exception that the rear portion of the housing 128 is formed free of opening 129 and instead defines a sealed surface such as, e.g., illustrated by the resorbable material applicator 100 embodiment shown in FIGS. 3A and 3B.

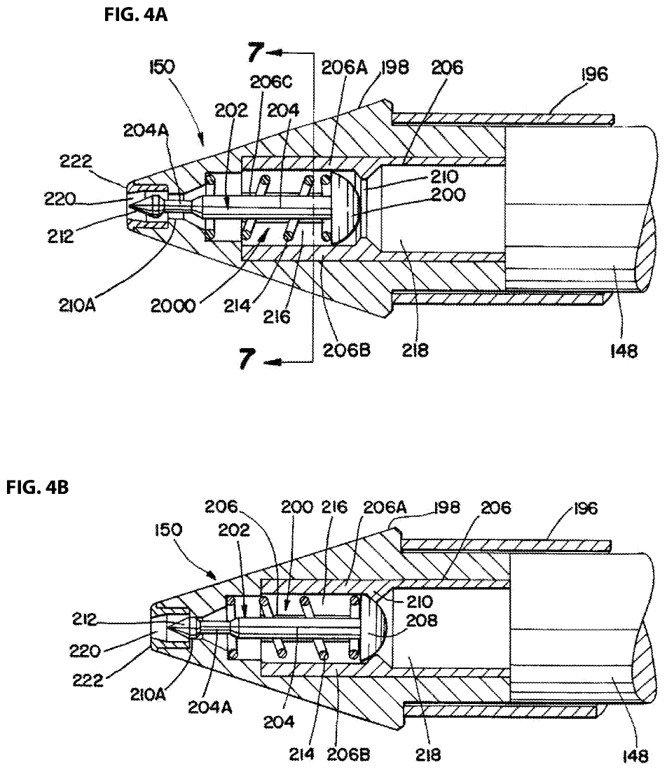

A detailed depiction of a nozzle 150 according to one embodiment in shown in FIGS. 4A and 4B. Nozzle 150 includes a body portion 151 and an insert portion 153, together defining a passageway 200 that extends through the nozzle 150 from a rear end of the nozzle 150 abutting the removable holder 196 front end, and a front end defining a dispensing opening. In some embodiments, such as illustrated e.g. in FIGS. 4A and 4B, the body portion 151 and insert portion 153 forming nozzle 150 may be defined by discrete elements, while in other embodiments (not shown) the body portion 151 and insert portion 153 may be defined by the same element, so as to define a monolithically formed nozzle 150.

Located within the passageway 200 is a valve assembly 202 including a valve stem 204 terminating at its rear end in a first valve head 208 that is configured to seat against a first valve seat 210 formed in the valve insert 206 when the valve assembly 202 is in the closed configuration illustrated in FIG. 4A and in which fluid flow through the nozzle is prevented. Located at the front end of the valve stem 204 is a second valve head 212 that is also configured to seat against a second valve seat 216 when the valve assembly 202 is in the closed configuration illustrated in FIG. 4A. Extending about the valve stem 204 between the first valve head 208 and the second valve head 212 is a valve bias spring 214 which is normally biased to the closed configuration of the valve assembly 202 illustrated in FIG. 4A.

Following activation on the driver motor 132 via a trigger 166 pressed by a user, the resorbable material stick 148 is urged (e.g. by feed roller 146, by a plunger, manually, etc.) through the heating element 136 and towards nozzle 150. After sufficient melted resorbable material from the resorbable material stick 148 has filled an upper portion 218 of the passageway 200 located upstream of the first valve seat 210 to overcome the bias of the valve bias spring 214, the first valve head 208 is unseated from the first valve seat 210 and the second valve seat 216 is unseated from the second valve seat 216, thereby allowing for flow of melted resorbable material through the passageway 200 and out from the nozzle 150. Such an open configuration of the valve assembly 202 is illustrated in FIG. 4B. Upon release of the trigger 166 by the user, movement of the resorbable material stick 148 is stopped, thereby minimizing the amount of melted resorbable material within the upper portion 218 of passageway 200, with the resultant decreased pressure of the melted resorbable material on the valve bias spring 214 causing the first valve head 208 to close the first valve seat 210 and the valve stem 204 to move the second valve head 212 so as to close the second valve seat 216, trapping substantially all of the melted resorbable material in the nozzle 150 within the passageway 200 until the trigger 166 is pulled again.

In some embodiments, it may be advantageous to prevent accidental over-delivery of resorbable material to the treatment site and/or it may be advantageous to control the maximum flow rate of melted resorbable material dispensed through the nozzle 150. Accordingly, in some embodiments (not shown), the valve assembly 202 may be modified to include features that allow for such volume and/or flow rate control. According to various embodiments, the resorbable material sticks 148 may alternatively, or additionally, be configured to allow a user to easily limit the volume of resorbable material that is to be delivered to the fracture site. For example, in some embodiments, resorbable material sticks 148 of different sizes corresponding to known, preselected volumes may be provided, such that a user may select a resorbable material stick size 148 having a volume appropriate to the fracture site being treated.

In other embodiments, the resorbable material sticks 148 may be formed of a plurality of detachable segments, with each segment corresponding to a known, preselected volume such that the user can easily, quickly and accurately adjust the size of the resorbable material stick 148 to correspond to a desired volume of resorbable material that is to be used to treat a fracture site without relying on any additional tools to do so. In such embodiments, adjacent segments may be separated by a weakening, formed as, e.g. a score, perforation, a thinned portion, etc. in the resorbable material stick 148 by which removal of individual segments of the resorbable material stick 148 may be facilitated.

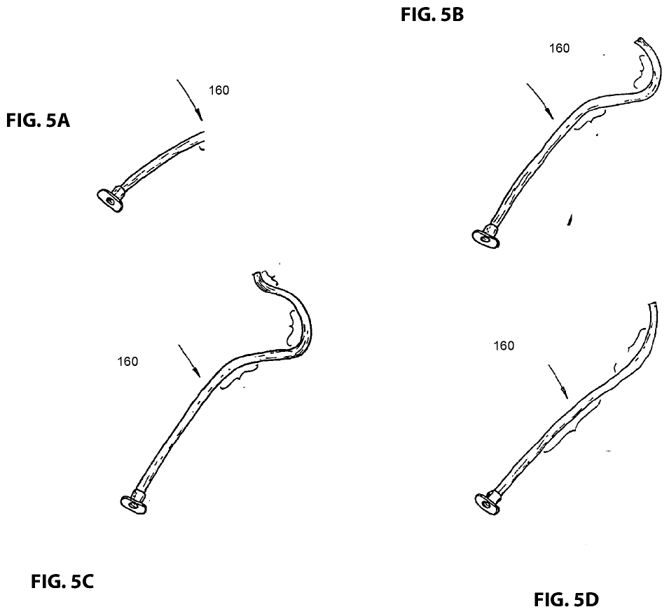

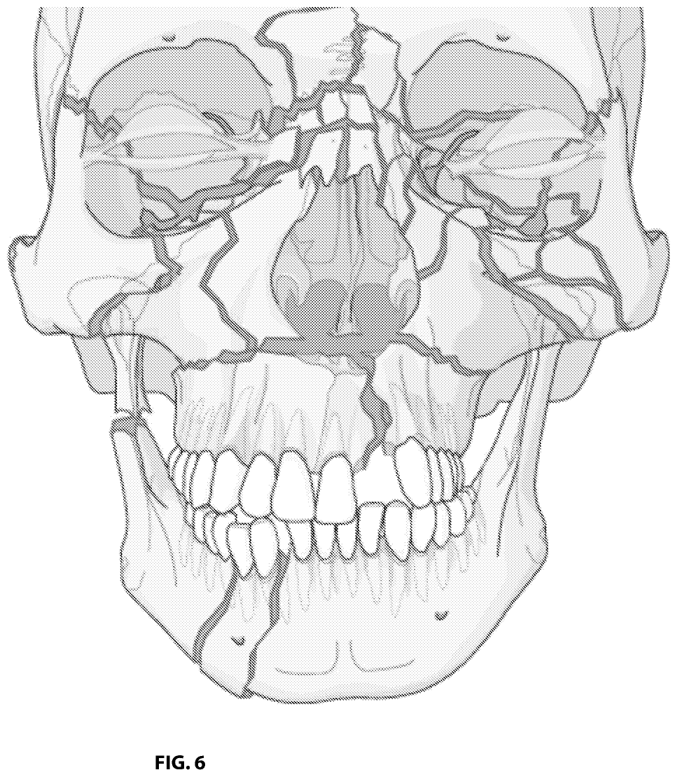

As will be described in detail below, given the small, tight spaces between adjacent bone fragments that the melted resorbable material is to be delivered to, the resorbable material applicator 100 may include a nozzle adaptor 160 configured to be attached to the nozzle 150 so as to provide the resorbable material applicator 100 with greater control and precision in dispensing melted resorbable material. Non-limiting examples of various nozzle adaptors 160 that may be attached to the nozzle are illustrated in FIGS. 5A-5D. Instead of nozzle adaptor 160 being separately provided from nozzle 150, it is to be understood that the nozzle 150 may be formed in the shape of any desired nozzle adaptor 160. The nozzle adaptor 160 may be defined by an outlet opening defining a cross-sectional area of between approximately 0.25 mm and approximately 5 mm, and more preferably between approximately 0.5 mm and approximately 1.0 mm. According to various embodiments, the nozzle adaptor 160 may be defined by a length of between approximately 10 mm and approximately 300 mm and more preferably between approximately 10 mm and approximately 100 mm.

Given the use of the resorbable material applicator 100 to apply melted resorbable material during treatment of a patient, it may be desirable to prevent the temperature of the melted resorbable material from exceeding a predetermined threshold temperature. Accordingly, in some embodiments, the resorbable material applicator may be provided with a temperature sensor, with the resorbable material applicator 100 being configured to prevent operation of the motor 132 in the event that a measured temperature of the melted resorbable material exceeds the predetermined threshold temperature.

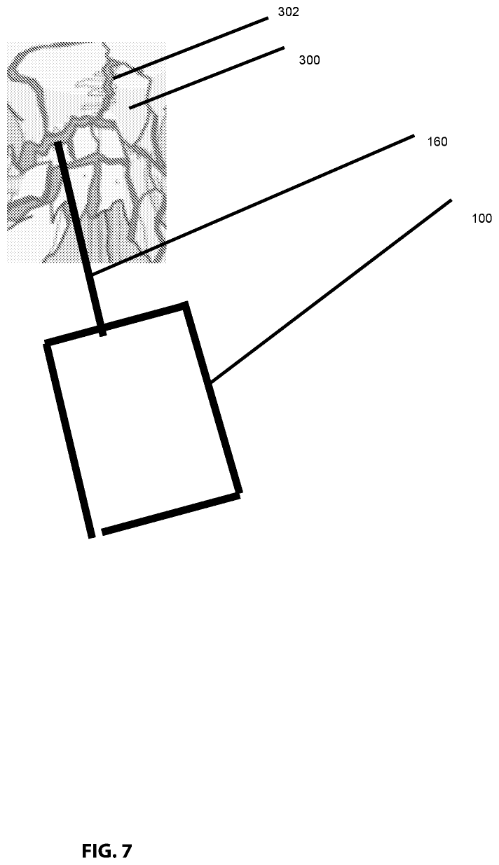

Referring to FIG. 6, an exemplary illustration of a patient exhibiting multiple craniomaxillofacial fractures is shown. According to conventional treatments, treatment of craniomaxillofacial fractures is based on the use of bone plates and screws which are used to connect adjacent bone fragments. However, in situations, such as, e.g. illustrated in FIG. 6, where the craniomaxillofacial trauma is convoluted and includes a fracture site defined by a significant amount of bone fragments, fragile bone fragments, and/or bone fragments of a small size, treatment of the fractures according to conventional bone plating methods may be difficult or not at all possible, as the fragments may not provide sufficient space to which the plates may be attached and/or the bone fragments may individually be too fragile to allow for attachment of a plate and screw. Treatment of craniomaxillofacial fractures in children, the elderly, or other individuals having thin, weak, or otherwise diminished bone strength and rigidity may pose similar issues to those obstacles faced in treating craniomaxillofacial fractures defined by a convoluted fracture pattern, as the individual bone fragments defining craniomaxillofacial trauma of such individuals may not have the requisite strength to structurally support an attachment of conventional bone plates and screws thereto.

A resorbable material applicator 100 such as described herein is advantageously configured to allow for craniomaxillofacial fracture stabilization without the risks and limitations of conventional bone plate methods. In particular, as illustrated in FIG. 7, the resorbable material applicator 100 allows for resorbable material to be directly applied to the edges 302 of bone fragments 300, allowing adjacent bone fragments 300 to be stabilized relative to one another. Unlike in the attachment of resorbable material bone plates, in which a heated resorbable material bone plate is placed over adjacent bone fragments, and to which an external pressure is directly applied (during e.g., contouring of the plate to the bone fragments and/or attachment of the plate to the bone fragments via screws) to effectuate a bonding between adjacent bone fragments, the dispensing of the melted resorbable material via an resorbable material applicator 100 allows adjacent bone fragments 300 to be bonded to one another. While in some embodiments a user may apply a gentle pressure or force to hold adjacent bone fragments 300 against one another as the applied melted resorbable material sets, according to other embodiments, the application of the melted resorbable material about the edges 302 of adjacent bone fragments 300 may adhere the adjacent bone fragments 300 to one another without requiring that any external force or pressure be applied directly to the bone fragment 300.

Additionally, because the nozzle 150 and optional nozzle adaptor 160 of the resorbable material applicator 100 allow for localized, direct placement of resorbable material to the fracture site, treatment of the fracture site with the resorbable material applicator 100 may be configured to reduce the need to create large incisions or openings in the patient as may otherwise be required for placement of the required bone plates and screws. Additionally, because resorbable material applied via the resorbable material applicator 100 may be applied locally to targeted areas of the bone fragments (such as, e.g. the edges 302 of bone fragments 300) as opposed to the general application of resorbable material bone plates atop larger portions of the exterior surface of adjacent bone fragments, the amount of resorbable material required to stabilize the fracture may be minimized.

Once the resorbable material that has been applied to the bone fragments 300 along the edges 302 has set (either with or without an externally provided user force or pressure being applied to hold the bone fragments in place during the setting or curing of the flowable resorbable material), the resultant interconnected bone fragments define a stabilized bone structure. In some embodiments, this stabilized bone structure may provide sufficient support such that further treatment of the fracture site may be postponed or altogether obviated. However, even in situations in which further treatment using conventional metal and/or resorbable material bone plates and screws is necessitated to further stabilized the fracutre, the structural integrity provided by the interconnected, resorbable material bonded stabilized bone structure may reduce the time and caution required to apply the bone plates and screws and/or may allow for such conventional bone plate treatment to be used in situations in which such treatments would previously have been unachievable (e.g. due to the extensive fracturing of the bone and/or the underlying weakness in bone strength of the patient).

Although the description above of the use of the resorbable material applicator 100 has made reference to the use of the resorbable material applicator 100 during the treatment of craniomaxillofacial fractures, it is to be understood that, according to various embodiments, the resorbable material applicator 100 may also advantageously be used in the treatment of bone fractures other than craniomaxillofacial bone fractures (such as, e.g., fractures of the bones of the legs, arms, ribs, etc.). In such embodiments, the resorbable material applicator 100 may optionally be modified to include a nozzle adaptor 160 shaped and sized to more easily apply flowable resorbable material to the specific bone fracture site being treated.

In embodiments in which the resorbable material applicator 100 is used to treat non-craniomaxillofacial fractures, the method of using the resorbable material applicator 100 may be generally similar to that as described with reference to treatment of craniomaxillofacial fractures, with the exception that, according to some such embodiments, following the application of flowable resorbable material using the resorbable material applicator 100, the subsequent stabilization of the non-craniomaxillofacial bone fracture may be accomplished using devices and/or methods (e.g. wires, external fixation devices, etc.) other than and/or in addition to the bone plates and screws conventionally used to stabilize craniomaxillofacial fractures.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of stabilizing a craniomaxillofacial fracture, the method comprising:
heating a resorbable material to make the resorbable material flowable;
applying the flowable resorbable material to a portion of a first bone fragment defining a fracture;
bonding the portion of the first bone fragment to an adjacent second bone fragment; and
stabilizing the first bone fragment relative to the second bone fragment.

2. The method of claim 1, wherein the first bone fragment is bonded to the second bone fragment by the applied resorbable material.

3. The method of claim 1 or claim 2, wherein the first bone fragment is stabilized relative to the second bone fragment by the applied resorbable material.

4. The method of claim 1 or any preceding claim, wherein the first bone fragment is bonded and stabilized relative to the second bone fragment without an external force being applied directly to either the first bone fragment or the second bone fragment.

5. The method of claim 1 or any preceding claim, wherein the first bone fragment is bonded and stabilized relative to the second bone fragment without the use of any fasteners or additional attachment elements.

6. The method of claim 1 or claim 2 or claim 3, further comprising attaching a bone plate to the first bone fragment and the second bone fragment.

7. The method of claim 6, wherein the first bone fragment is stabilized relative to the second bone fragment by the attached bone plate.

8. The method of claim 7, wherein the bone plate is attached to the first bone fragment by a first screw.

9. The method of claim 8, wherein the bone plate is attached to the second bone fragment by a second screw.

10. The method of claim 1 or any preceding claim, wherein the resorbable material is applied via a resorbable material dispensing applicator to an edge of the first bone fragment.

11. The method of claim 10 or any preceding method claim, wherein the dispensing applicator comprises a feeder element configured to move a solid piece of a resorbable material past a heating element and into a nozzle.

12. The method of claim 11, wherein the feeder element is configured to move the resorbable material in response to the activation of the dispensing applicator via a trigger.

13. The method of claim 11 or claim 12, wherein the nozzle includes a valve assembly configured to prevent flow of melted resorbable material when the trigger is released.

14. A method applying, a resorbable material to a craniomaxillofacial fracture, the method comprising:
providing a resorbable material dispensing applicator comprising a housing body within
which is located:
a motor;
a drive element configured to be activated by the motor;
a feeder element configured to move in response to activation of the drive element; and
a heating element;
loading a solid piece of resorbable material into the dispensing applicator;
activating the feeder element to translate the solid piece of resorbable material relative to the heating element to melt the solid piece of resorbable material; and
dispensing melted resorbable material through a nozzle of the dispensing applicator to a first bone fragment.

15. The method of claim 14 or any preceding method claim, wherein the applied melted resorbable material adheres the first bone fragment to an adjacent bone fragment.

16. The method of claim 15 or any preceding method claim, wherein the melted resorbable material is applied to an edge of the first bone fragment.

17. The method of claim 14 or of any preceding method claim, wherein the applicator is reusable.

18. The method of claim 14 or claim 15 or claim 16 or claim 17, wherein the feeder element is configured to rotate in response to activation of the drive element, the rotation of the feeder element configured to urge the solid piece of resorbable material towards the heating element and nozzle.

19. The method of claim 14 or claim 15 or claim 16 or claim 17 or claim 18, wherein the dispensing applicator further comprises a pinch roller configured to urge the solid piece of resorbable material into contact with the feeder element.

20. The method of claim 14 or any preceding method claim, wherein the resorbable material comprises one of poly-D and L-lactic acid, L-lactide-co-glycolide, an admixture of polylactic acid, and an admixture of polyglycolide acid.

21. A resorbable biomaterial dispensing applicator configured to dispense melted resorbable biomaterial to a craniomaxillofacial fracture, the applicator comprising:
a housing body;
a motor;
a drive element configured to be activated by the motor;
a heating element defining a passageway therethrough, the heating element configured to melt a resorbable material stick;
a feeder element configured to move in response to activation of the drive element, the movement of the feeder element configured to translate the resorbable material stick into the passageway of the heating element; and
a nozzle defining a passageway extending between an outlet of the heating element passageway and an outlet opening, the nozzle being defined by a cross-sectional area of between approximately 0.25 mm and approximately 5 mm and a length of between approximately 10 mm and approximately 300 mm, wherein the nozzle is configured to allow the heated and melted resorbable material to be applied to an edge of a bone fragment forming a part of the craniomaxillofacial fracture.

22. The resorbable biomaterial dispensing applicator of claim 21, further comprising a resorbable material stick loaded within the housing body.

23. The resorbable biomaterial dispensing applicator of claim 21 or 22, further comprising a temperature sensor, the temperature sensor configured to prevent movement of the feeder element in response to a sensed temperature, the melted bioresorbable material within the housing body being greater than a predetermined threshold temperature.

24. The resorbable biomaterial dispensing applicator of claim 18, wherein the resorbable material stick is entirely enclosed by the housing body when the resorbable material stick is loaded within the housing body.
